(19) United States
(12) Patent Application Publication
(10) Pub. No.: US 2018/0014546 A1
(43) Pub. Date: Jan. 18, 2018

(54) Broad Spectrum Bacteriocin for Control of Unwanted Bacteria

(71) Applicants: Elizabeth J. Summer, College Station, TX (US); Mei Liu, College Station, TX (US)

(72) Inventors: Elizabeth J. Summer, College Station, TX (US); Mei Liu, College Station, TX (US)

(21) Appl. No.: 15/715,582

(22) Filed: Sep. 26, 2017

Related U.S. Application Data
(63) Continuation-in-part of application No. 14/046,430, filed on Oct. 4, 2013.

(51) Int. Cl. A01N 63/02; A23L 3/3463; A23C 19/11; C12P 7/06; A23C 9/158

(52) U.S. Cl. CPC A01N 63/02; C12P 7/06; A23C 9/1585; A23C 19/11; A23L 3/3463

(57) ABSTRACT

A composition containing a newly identified bacteriocin produced by a Lactobacillus plantarum strain, isolated from natural corn mash used in commercial fermentation process for production of ethanol. The bacteriocin composition is effective in broad range killing against the vast majority of the lactic acid bacteria (LAB) isolates in ethanol fermentation mash, which include multiple species of Lactobacillus, Lactococcus, Weissella, Leuconostoc, Pediococcus, Enterococcus, and Streptococcus as well as killing activity against non-LAB isolates, such as strains of Staphylococcus, Enterobacter, Bacillus, and Clostridium. The bacteriocin is also combined with broad range bacteriophage to provide synergistic effectiveness again unwanted bacteria in industrial biofuel fermentation process and in human and animal bacterial infections.

### CROSS REFERENCE TO RELATED APPLICATIONS

This application is a continuation-in-part application of U.S. ser. No. 14747 filed Jan. 5, 2014 which application claims benefit of Provisional Application Ser. No. 61/991,034 filed May 9, 2014, the content, figures and disclosures of which are incorporated herein by reference.

### BACKGROUND

### Field

A broad spectrum bacteriocin composition and method for control of unwanted bacteria, particularly lactic acid bacteria found in fermentation processes.

### Background

Bacteriocins are usually antimicrobial peptides produced by bacteria, particularly Gram-positive bacteria, to inhibit the growth of related species or other genera at high potency. Lactic acid bacteria (LAB) are a group of popular bacteriocin-producing bacteria, with a substantial number of bacteriocins produced by LAB strains discovered and well characterized. The characterized bacteriocins produced from LAB can be classified into three major groups: Small and heat stable bacteriocins containing lanthionine (class I bacteriocin, or lantibiotics), small and heat-stable non-lanthionine-containing bacteriocins (Class II), and large and heat-labile lytic protein (Class III).

Antimicrobial spectrums of bacteriocins produced by LAB can vary depending on their specific protein structures. Most of the LAB bacteriocins are capable of inhibiting a wide range of LAB species/strains, and some of LAB bacteriocins are also very potent against some Gram-negative bacteria. Bacteriocins showing broad activity spectrum against target bacteria populations can have wide application for industrial antimicrobial use and remediation of human and animal bacterial infection. For example, plant material-based commercial ethanol fermentation process often suffers from LAB contamination and therefore the inhibition of yeast activity, which results in significant product yield loss. The indigenous contaminating LAB population in the fermentation raw materials is control targets since they pose potential risk on yeast fermentation. Bacteriocins produced by the indigenous LAB population are more likely to act towards species and/or strains of the indigenous LAB population and thus can be used as an effective control on contaminating LAB. The contaminating LAB population in ethanol plants has been identified to contain multiple LAB genera and each LAB genus contains different species. A bacteriocin with broad LAB control spectrum is needed for such contamination control. Bacteriocins effective for broad-spectrum LAB remediation could also be effective against LAB species and/or other pathogenic agents associated with dental caries, bovine mastitis and other clinical infections.

In addition the use of bacteriocin derived from lactic acid bacteria as antimicrobials is safe. Bacteriocins from LAB are described as “natural” inhibitors, in regard to the long history of safe use of LAB in food industries. Lactic acid bacteria are ubiquitous in nature and are commonly associated with plant materials and thus commonly present in fermented food. Many bacteriocins produced by lactic acid bacteria are isolated from foods such as meat and dairy products, which normally contain lactic acid bacteria. These bacteriocins have been unknowingly consumed by human for centuries. Some of the broad spectrum bacteriocins produced by LAB are used commercially in food and pharmaceutical industries. Examples include nisin (a Class Ia bacteriocin) produced by Lactococcus lactis and pediocin (Class II bacteriocin) produced by Pediococcus acidilactici. Nisin is granted GRAS (generally recognized as safe) status by FDA (GRAS Notice No. GRN 000065). It is approved for use in over 40 countries and has been used as food preservatives for over 50 years.

The present invention is a composition of a newly identified broad range bacteriocin produced by a Lactobacillus plantarum strain, isolated from natural corn mash used in fermentation process for production of ethanol.

### SUMMARY

This invention is a composition of a newly identified bacteriocin produced by a Lactobacillus plantarum strain, isolated from natural corn mash used in commercial fermentation process for production of ethanol. The bacteriocin composition is effective in broad range killing against the majority of the LAB isolates in ethanol fermentation mash, which include multiple species of Lactobacillus, Lactococcus, Weissella, Leuconostoc, Pediococcus, Enterococcus, and Streptococcus as well as killing activity against non-LAB isolates, such as strains of Staphylococcus, Enterobacter, Bacillus, and Clostridium. The bacteriocin is also combined with broad range bacteriophage to provide synergistic effectiveness again unwanted bacteria in industrial biofuel fermentation process and in human and animal bacterial infections.

### DESCRIPTION OF FIGURES

FIG. 1 is a schematic representation of the proteins responsible for producing the bacteriocin of the invention. The arrangements of the proteins are consistent with their positions on the bacterial host genome.

FIG. 2 is a representation of result of a test showing the reduction of indigenous contaminating bacterial level in fermentation mash from a commercial ethanol plant by the bacteriocin of this invention designated for identification as GP15cin.

FIG. 3 is a representation of result of a test showing the reduction of indigenous contaminating bacterial level in fermentation mash from a commercial ethanol plant by the bacteriocin of this invention designated for identification as GP15cin.

FIG. 4 is a representation of result of a test showing the reduction of indigenous contaminating bacterial level in fermentation mash from a commercial ethanol plant by the bacteriocin of this invention designated for identification as GP15cin.

FIG. 5 is a representation of result of a test showing the reduction of indigenous contaminating bacterial level in fermentation mash from a commercial ethanol plant by the bacteriocin of this invention designated for identification as GP15cin.

FIG. 6 is a representation of result of a test showing the reduction of indigenous contaminating bacterial level in fermentation mash from a commercial ethanol plant by the bacteriocin of this invention designated for identification as GP15cin.

FIG. 7 is a representation of result of a test showing the reduction of indigenous contaminating bacterial level in fermentation mash from a commercial ethanol plant by the bacteriocin of this invention designated for identification as GP15cin.

FIG. 8 is a representation of result of a test showing the reduction of indigenous contaminating bacterial level in fermentation mash from a commercial ethanol plant by the bacteriocin of this invention designated for identification as GP15cin.

FIG. 9 is a representation of result of a test showing the reduction of indigenous contaminating bacterial level in fermentation mash from a commercial ethanol plant by the bacteriocin of this invention designated for identification as GP15cin.

FIG. 10 is a representation of result of a test showing the reduction of indigenous contaminating bacterial level in fermentation mash from a commercial ethanol plant by the bacteriocin of this invention designated for identification as GP15cin.

FIG. 11 is a representation of result of a test showing the reduction of indigenous contaminating bacterial level in fermentation mash from a commercial ethanol plant by the bacteriocin of this invention designated for identification as GP15cin.

FIG. 12 is a graphical representation of the synergistic effects of a combination of the bacteriocin of the invention and a bacteriophage composition in reduction of LAB.

### DETAILED DESCRIPTION

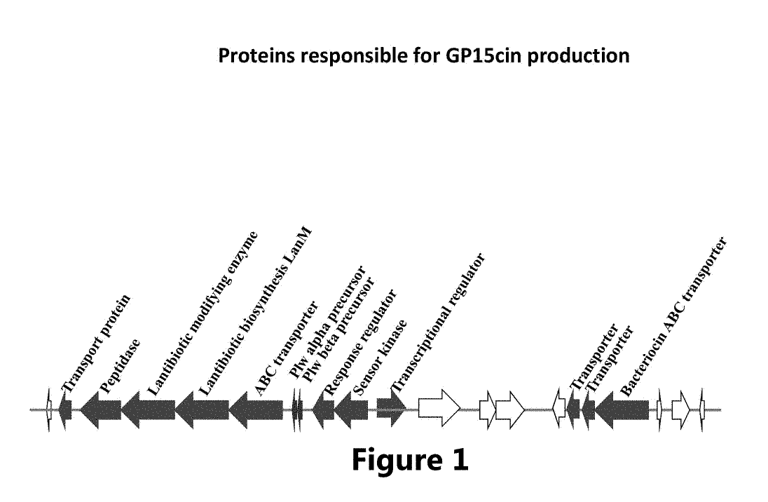

This invention is a composition comprising a specific broad spectrum bacteriocin, for control of unwanted bacteria, particularly lactic acid and other acid producing bacteria found in fermentation processes. FIG. 1 is a schematic representation of the proteins responsible for producing the bacteriocin of the invention. The arrangements of the proteins are consistent with their positions on the bacterial host genome. Additionally, methods of making and using the composition are disclosed. In another embodiment, the bacteriocin of the invention combined with bacteriophage is applied to achieve a synergistic reduction of unwanted lactic acid bacteria (LAB) and other bacteria.

Unwanted bacteria as the term is used herein are bacteria that are unwanted or harmful in a specific environment such as lactic acid and other acid producing bacteria that produce acids that interfere with fermentation to ethanol processes and bacteria that cause bacterial infection in human and animals and bacteria that produce harmful biofilms. They are strain(s) of bacteria specifically targeted for control. The unwanted bacteria need not necessarily be known, isolated, or identified; the sole defining characteristic is that it is the organism(s) desired to be controlled.

### Bacteriocin Isolation and Characterization

### (1) Bacteriocin Isolation

In the discovery of the unique broad spectrum bacteriocin of this invention, indigenous LAB were isolated and purified from the fermentation mash of the commercial ethanol fermentation plants. The identities of the isolates were determined to the species level based on their 16s rDNA sequences. The capability of the isolated LAB strains to produce antimicrobial compounds was identified. Culture supernatants of the strains to be tested were spotted onto individual indicator bacterial culture lawns, which were prepared using a variety of LAB strains of different genera. After incubation, growth inhibition of the indicator bacteria was seen as a clear zone at and around the spotting position. Any positive activity of bacterial growth inhibition from the tested culture supernatant was confirmed again in the same manner, and the inhibitory activity spectrum of the antimicrobial compound is determined. One of the identified antimicrobial compounds is produced by a Lactobacillus plantarum strain, designated for identification as GP15, and this bacteriocin is similarly named GP15cin for easy identification. GP15cin was shown to exhibit a broad range killing activity against the vast majority of the LAB isolates from commercial ethanol plants, which include multiple species of Lactobacillus, Lactococcus, Weissella, Leuconostoc, Pediococcus, Enterococcus, and Streptococcus. GP15cin also showed killing activity against non-LAB isolates, such as strains of Staphylococcus, Enterobacter. Bacillus, and Clostridium.

### (2) Bacteriocin Extraction and Concentration

Bacteriocin molecules produced by the host bacteria can be extracted, concentrated, and then purified by a variety of different strategies. For example, GP15cin can be precipitated out from the bacterial culture supernatant by using ammonium sulfate “salting out” method, where a desired saturation percentage of salt is reached by adding ammonium sulfate slowly to the cell-free bacteriocin-containing culture supernatant. After incubating the salt suspension agitated overnight at 4° C., the salted-out proteins were precipitated by centrifugation and dissolved in a small volume of appropriate buffer, such as phosphate buffer (10 mM, pH 7.0). The precipitated mixture can be desalted by dialysis with membrane(s) of appropriate molecular weight cut-off. Bacteriocin molecules in the cell-free bacterial supernatant can also be extracted using organic solvents, such as cold acetone or chloroform. The bacteriocin-solvent mixture can be separated from the aqueous supernatant layer via centrifugation. The solvent in the bacteriocin-solvent mixture can be evaporated to concentrate the extracted bacteriocin molecules, which exist in the peptidic fraction. The extracted and concentrated bacteriocin can then be used for characterization study.

### (3) Bacteriocin Purification

The extracted and concentrated bacteriocin molecules can be purified, typically by chromatography-based methods. Based on the knowledge of the target bacteriocin characteristics such as its estimated size, molecule net charge at a definite pH, adsorption affinity, molecule polarity and hydrophobicity, etc., different separation strategies can be used. The strategies include size exclusion, ion exchange, gel filtration, hydrophobic interaction, reverse phage liquid chromatography, etc. For example, the bacteriocin extract can be passed through a size exclusion chromatography separation column and the mixture can be separated into fractions of different molecule sizes. Ion exchange chromatography, using either cation or anion exchange columns, can also be used to separate the bacteriocin extracts based on their electric charge at a definite pH. In addition to the conventional chromatography columns, centrifuge-based protein separation cartridges are commercially available and can also be used for bacteriocin purification. Multiple chromatography-based strategies can be combined for optimal separation. To locate the bacteriocin molecules, the separated fractions can be tested for their antimicrobial activities by spotting onto indicator bacteria lawns.

### (4) Bacteriocin Production Kinetics

Bacteriocin production is dependent on the host growth, and the production kinetics can be determined by quantifying the bacteriocin levels at different stages of host growth. The bacteriocin production in Lactobacillus sp. strain GP15 appear to start when the host cells reach late-logarithmic and early stationary growth phase, and after that a rapid production rate is observed and the maximum level of bacteriocin is reached when host cells enter mid-stationary growth phase. The optimal production of GP15cin is subject to nutrient regulation, and the optimal production medium with optimal levels of nitrogen and carbon sources has been developed.

### (5) Bacteriocin Activity Range

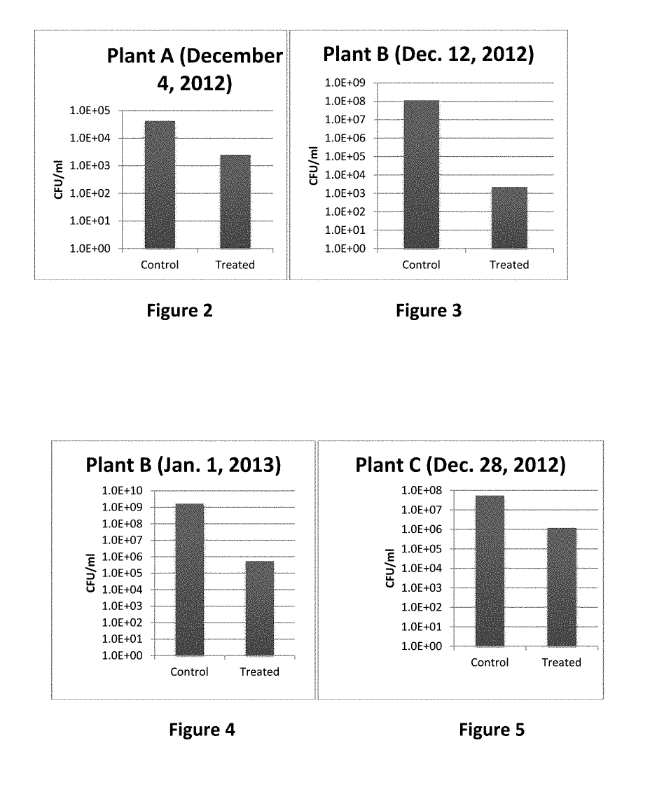

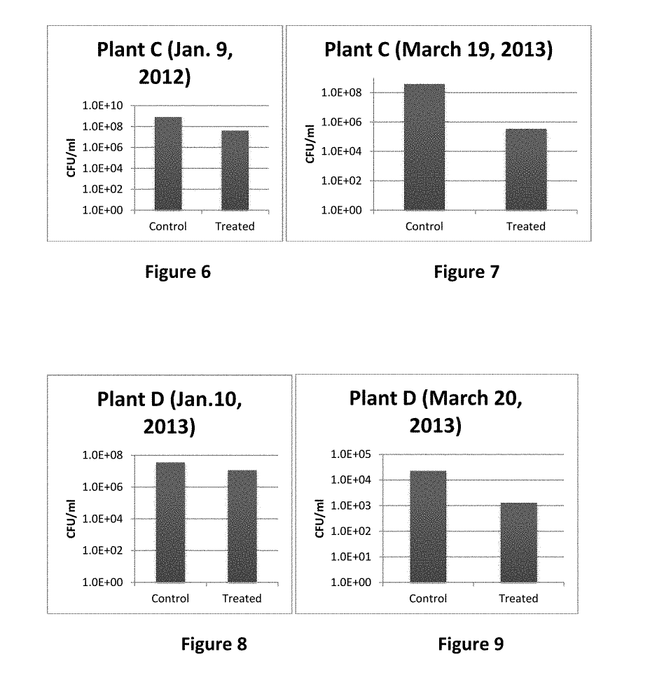

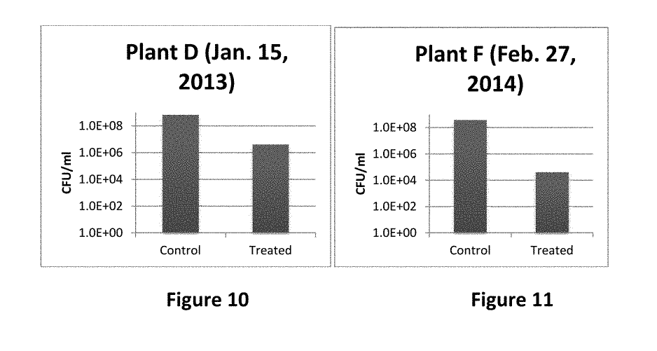

The inhibitory activity spectrum of GP15cin against different strains was determined. The active concentration range and the minimum inhibition concentration of the bacteriocin are determined using different indicator strains. The activity levels of the bacteriocin present in the raw bacterial culture supernatants and/or in partially purified mixtures were also determined. GP15cin showed broad killing activity against the majority of the LAB isolates from commercial ethanol plants, which include multiple species of Lactobacillus, Lactococcus, Weissella, Leuconostoc, Pediococcus, Enterococcus, and Streptococcus. GP15cin also showed killing activity against non-LAB isolates, such as strains of Staphylococcus, Enterobacter, Bacillus, and Clostridium. FIG. 2 to FIG. 11 show the effectiveness of GP15cin in reducing the total levels of indigenous bacteria presented in fermentation mash from different commercial ethanol plants. The exact identities of the indigenous bacteria population in different plants are unknown and are likely highly diverse. The consistent reduction effects seen in all tests highlight the broad activity range of GP15cin.

### (6) Bacteriocin Chemical Characteristics

Either using crude, partially or completely purified form, the chemical characteristics of the bacteriocins can be determined. The determined characteristics include their sensitivities to different enzymes, stability at different temperatures and pH values, etc. Sensitivity of the bacteriocin to proteolytic and lipolytic enzymes, such as trypsin, pepsin, pronase, lipase, etc, was tested. The proteinaceus nature of GP15cin was confirmed. It was determined that GP15cin has 50% reduction in activity at temperatures of 65-100° C. with a 87.5% reduction seen at a temperature of 120° C. However, there was not a complete loss of activity seen even after 2 hours at 120° C., indicating GP5cin is heat resistant. The same bacteriocin has optimal activity in the pH range of 4-10. There is a complete loss of activity at pH 12, and there is a 50% reduction in activity at pH values of 1, 2, 3, and 11. The activity loss at the extreme pH values does not appear to be reversible.

### (7) Bacteriocin Coding DNA Sequence and Protein Structure Identification

Once the bacteriocin is purified to homogeneity, its molecular weight, amino acid sequence and protein structure can be determined with precision using standard chemical analysis methods such as mass spectrometry and nuclear magnetic resonance (NMR) spectroscopy. The genomic DNA of the host bacterial strain can be extracted, and the genome of the bacteria can be sequenced. The DNA sequences obtained can be compared to that of previously identified bacteriocins, to elucidate the genes encoding the bacteriocins and related proteins. We sequenced the genome of the GP15cin producing strain, L. plantarum GP15. The genome sequencing generated 97 contigs encoding 3255 genes in total. The genome size is approximate 3.2 million base pairs, and the GC content of the genome is approximate 44%. The obtained genome sequence provided clear characterization of the bacteriocin. A two-peptide lantibiotic locus was identified on Contig 31. There are 21 genes identified in the locus and their coded proteins are illustrated in FIG. 1. Bacteriocin precursor proteins are the Plw alpha precursor and Plw beta precursor. Proteins with other known functions, including transporter proteins, transcriptional regulator proteins, and different enzymes, are labeled over proteins in FIG. 1. Proteins with unknown functions are those not named in FIG. 1.

The nucleotide sequences of the region containing the structural genes of GP15cin are identical to that of plantaricin W identified in L. platarum LMG 2378 (GenBank accession number AY007251. Holo H, Jeknic Z, Daeschel M, Stevanovic S, Nes I F.; Plantaricin W from Lactobacillus plantarum belongs to a new family of two-peptide lantibiotics. Microbiology. 2001. 147(Pt 3): 643-51). The plantaricin-producing strain, Lactobacillus plantarum LMG 2379, was isolated from fermenting Pinot Noir wine in Oregon. We conclude that plantaricin W (Plw) is the sole or major component of GP15cin. GP15cin, or plantaricin W, consist of two peptides, Plwα (comprising 29 residues) and Plwβ (comprising 32 residues). Like other two-peptide bacteriocins studied, the two structural genes encoding these two peptides are located next to each other as a transcriptional unit. A promoter was identified upstream of these two structural genes. Chemical analyses of plantaricin W showed that both peptides (plwα and Plwβ) are lantibiotics, but two unmodified cysteines and one serine residue were present in Plwα and Plwβ contained one cysteine residue. The individual peptides act synergistically, with each individual peptide showing low antimicrobial activities when acting alone. Thus, GP15cin can be defined herein and in the claims as a two-peptide lantibiotic consisting of the peptide Plwα, comprised of 29 residues, and the peptide Plwβ, comprised of 32 residues, with the genes encoding the precursor proteins being located next to each other in a transcriptional unit that includes additional genes required for lantibiotic biosynthesis, and neither the genes nor the amino acid sequences sharing any similarity to Nisin. The peptides are initially synthesized as linear molecules and are post-translationally modified such that the mature, active peptide is polycyclic. The two subunits are post-translationally modified to include α-aminobutyric acid, 3-methyllanthionine, lanthanide, 2,3-diehydroalanine, 2,3-didenydrobutyrine, and pryuvic acid derived from deamination of Dha residues as well as cysteine-cysteine disulfide bonds.

Additionally, GP15cin may be characterized as a two-peptide lantibiotic bacteriocin in which the protein sequence responsible for its production as shown schematically in FIG. 1.

In addition to plantaricin W in L. platarum LMG 2378, the peptides of GP15cin also share significant sequence similarities with other two-peptide lantibiotics. These include mersacidin from Bacillus halodurans (GenBank accession number NP_241320.1), enterocin W from Enterococcus faecalis (GenBank accession number BAL50001.1), lacticin 3147 from L. lactis, and staphylococcin C55 from S. aureus, etc.

The bioinformatic analysis of the host genome sequence clearly characterized GP15cin as a two-peptide lantibiotics. To be specific, GP15cin was identified as plantaricin W. Together with previously identified ones, GP15cin belongs to Class I bacteriocins (lantibiotics). Different from polycyclic lantibiotics (such as nisin) that are classified as Class Ia, these two-peptide lantibiotics are linear in structure and are classified as Class Ib.

### Bacteriocin Production for Useful Applications

Bacteriocins need to be produced at a large scale for commercial application. The produced bacteriocin can be used either in crude preparations or purified forms. Bacteriocins can be produced in several ways: (1) using bacteriocin-producing strains directly; (2) using recombinant protein over-expression systems. Alternatively, bacteriocin may be produced in situ utilizing the above-mentioned systems during the treatment process.

### (1) Bacteriocin Production in Native Host Strains

The culturing conditions of the bacteriocin producing strain are first optimized in a small volume batch culture to achieve the maximum yield of bacteriocin production. The optimized parameters include the media composition, culture temperature and pH, anaerobic conditions, etc. In addition to maximizing bacteriocin yields, the optimized parameters will also consider the culture volume scale up and downstream concentration and purification of bacteriocin if applicable. Production of bacteriocin at a large scale will be achieved in large volume culture vessels or commercial fermentors. Bacteriocin production can also be achieved in fed-batch cultures where the determined limiting nutrient substrates are fed to the culture to sustain the high level bacteriocin production for a longer time.

It is possible to obtain bacteriocin over-producing mutants of the host strain via natural mutation events. With the identification of the genetic determinants of bacteriocins through genome sequencing, it is possible to obtain bacteriocin over-producing mutants of the host via specific genetic manipulations. The optimal growth conditions of these bacteriocin over-producing mutants can be determined for bacteriocin production at a much higher level.

### (2) Bacteriocin Over-Expression Using Non-Native Expression Hosts

FIG. 1 is a schematic representation of the proteins responsible for producing the bacteriocin of the invention. The structural genes directly encoding GP15cin are located in the same cluster as other genes involved in production, such as regulatory, modification and transporter genes. The identified GP15cin genetic determinants can be cloned into plasmid expression vectors, and the vectors can be introduced into bacterial hosts different from the native bacteriocin-producing strains. There are commercially available over-expression systems using well-characterized expression hosts, and the protein expression techniques in those hosts are well developed. Many developed expression systems are also compatible with downstream protein purification and large-volume scaling up. The expression vectors can also be bacteriophage-based. It is possible to engineer bacteriophage to carry the bacteriocin genetic determinants, and the expression of bacteriocin will be accomplished replying on phage proliferation cycles.

Either using the native bacteriocin-producing strains or using recombinant protein over-expression systems in non-native strains, bacteriocin produced can be used in its crude forms for application. The bacteriocin produced can also be concentrated and purified for application. Alternatively, bacteriocin-producing strains may be introduced to the systems to be treated, and the bacteriocin of interest may be produced in situ utilizing the above-mentioned systems during the treatment process.

Bacteriocin can be used as the only active ingredient for anti-microbial application. Alternatively, bacteriocin can be used together with other agents or adjuvants for enhanced activity. For example, bacteriocin can be used together with bacteriophage to achieve broader range of control and with greater effects.

### Bacteriocin Applications

Either used alone or in combination with other antimicrobial agents, the two-peptide lantibiotic GP15cin described herein can be used in the following practices where either specific or general lactic acid bacteria are the controlling targets:

### Biofuel Fermentation Process

GP15cin and other bacteriocins targeting LAB can be used to control bacterial contamination in various biomass-refining processes, including but not limited to ethanol biofuel production. Commercial ethanol plants use starch or sugar-based raw plant materials and rely on yeast fermentation for ethanol production. This microbiological process uses non-sterile substrates and thus is inevitably subjected to bacterial contamination, often to the detriment of fermentation performance ultimately resulting in significant ethanol yield loss. Lactic acid bacteria, especially Lactobacillus spp., are the predominant contaminating bacterial group identified worldwide. Common approaches used by fermentation plants to reduce LAB contamination include sanitization and the copious use of antibiotics such as virginiamycin and penicillin. Compared to current controlling products, bacteriocins originated from LAB are of GRAS (generally recognized as safe) status as designated by the U.S. Food and Drug Administration (FDA) and pose no environmental hazards.

GP15cin described herein were produced by a L. plantarum strain, which was isolated from the indigenous LAB populations present during commercial ethanol fermentation. GP15cin can act towards species and/or strains of similar indigenous LAB populations and thus can be used as an effective control for contaminating LAB. FIGS. 2-11 shows the reduction of indigenous contaminating bacterial levels in ethanol fermentation mash by GP15cin. Either fermentor or mash cooler mash samples were collected from 6 different commercial ethanol plants on 13 different days from 2012 to 2014. Results of 15 independent tests are shown. When used in its crude form, GP15cin reduced indigenous contaminating bacteria levels in all mash samples. The reduction magnitude was as much as 99.99999% (>800,000 fold), and the reduction effect was seen in as little as 2 hours. In addition, GP15cin significantly reduced the lactic acid and acetic acid levels produced by contaminating LAB during laboratory-scale ethanol fermentation. It is likely that the effects on controlling contaminating bacteria and acids will be even more significant if a higher dose of GP5cin is used. The presented results highlight the feasibility of GP15cin application for control of unwanted bacteria in ethanol and other biofuel industries.

The application of bacteriocin and/or bacteriophage to control LAB in common ethanol production plants using starch feeds is well documented in the patent literature including published patent applications 2014/0148379, published May 29, 2014 and 2009/0104157, published Apr. 23, 2009, the disclosure and figures of which are incorporated herein by reference. Application of the present invention bacteriocin GP-15cin can be utilized in the same way as described in these applications.

As noted above, GP-15cin may be applied alone or in combination with other bacteriocin and/or bacteriophage virulent for unwanted bacteria in the same system. Bacteriocin producing hosts are immune to the bacteriocins produced by themselves. GP-15cin is therefore not active against its own production host, GP15cin. Though the bacteriocin produced by Lactobacillus GP15 exhibits broad host range in inhibiting various indigenous LAB, a very small number of strains of LAB present in natural fermentation mash may not be sensitive to this bacteriocin. Therefore, it is desirable to combine GP-15cin with a different bacteriocin in a cocktail that will kill the bacteria insensitive to GP15cin. Additionally, GP-15cin may be combined with other antimicrobial agents to broaden the scope of effectiveness against the range of unwanted bacteria that are targeted. For example, the combination of bacteriocin and bacteriophage (phage) ensures a broader inhibition spectrum and more efficient control of the bacteria contamination. Phage targeting these non-sensitive strains can be isolated, and be combined with the bacteriocin for a complete control of contaminating bacteria.

### The Synergistic Effect Between Bacteriocin and Bacteriophage

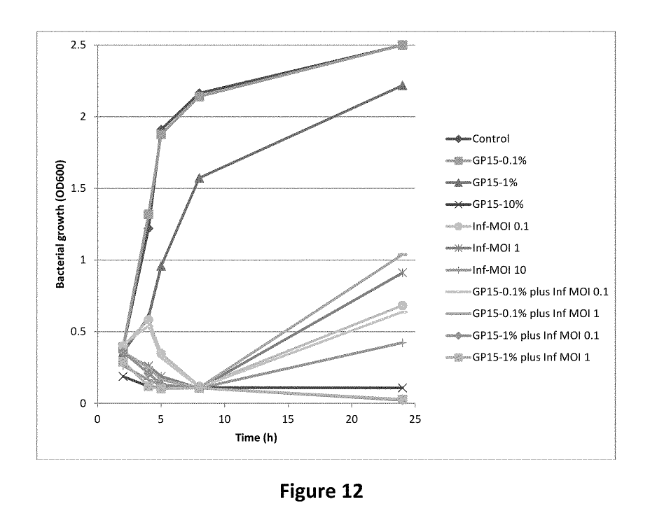

The combination of the GP-15cin bacteriocin and some bacteriophage has been found to have a synergistic effect in remediation of unwanted LAB bacteria isolated from ethanol fermentation plants. Bacteriocin alone, bacteriophage alone, and bacteriocin combined with phage were tested against Lactobacillus in batch culture systems. To be specific, logarithmic growth phase cultures of Lactobacillus fermentum strain 0315-25 (OD600 being 0.1) were treated either with single agent at different doses, such as bacteriocin GP15cin alone at different doses (0.1%, 1%, and 10%, v/v), or a phage designated herein as LferInf alone at different MOIs (0.1, 1, and 10), or two agents combined together at different doses, such as 0.1% GP15cin plus LferInf at MOI of 0.1, 0.1% GP15cin plus phage LferInf at MOI of 1, 1% GP15cin plus LferInf at MOI of 0.1, and 1% GP15cin plus phage LferInf at MOI of 1. Culture without any treatment was served as control. LferInf is a bacteriophage isolated from wastewater and found to be effective against a broad host range of Lactobacillus and described in more detail below. The growth of bacteria was monitored for 24 h, and the treatment effects were compared. The results are shown in FIG. 12. Compared to the control, all treatment, except GP15cin used at 0.1%, suppressed the growth of L. fermentum. Among the treatments, the greatest growth inhibition effects were observed from the bacteriocin and phage combinations, with each agent used at a lower dose (1% GP15cin plus LferInf at MOI of 0.1, and 1% GP15cin plus LferInf at MOI of 1). The combinational effects were comparable or greater than the single agent used at its highest dose, i.e. GP15cin at 10%, or phage at MOI of 10. There is therefore an obvious synergistic effect between GP15cin and the LferInf when used together.

This synergistic effect between two antimicrobial agents in inhibiting bacterial growth has great application potentials. This synergistic effect between bacteriocin and phage allows effective suppression of target bacteria, as well as significantly reduced production cost, since the synergistic effect allows each agent to be used at a much lower dose than that required when a single agent is used. Bacteriocin and phage can be added to ethanol fermentation at the same time, or added sequentially depending on the growth kinetics of their respective target hosts. Bacteriophage virulent for LAB bacteria useful for this invention may be identified, isolated, purified, encapsulated, and commercially produced by means and methods known in the art. See for example U.S. application Ser. No. 13/465,700 filed May 7, 2012, now published application US 2013/0149753; U.S. application Ser. No. 13/466,272 filed May 8, 2012, now published application US 2013/0149759; Published application US 2009/0104157, published Apr. 23, 2009 and WO 2006/050193. The disclosures of these patents and patent applications are incorporated herein by reference for all purposes. In this invention any of the bacteriophage virulent for LAB found in the fermentation of starches and sugars to biofuel processes may be used, as well as bacteria that produce the phages in situ.

In general it is not preferred to add the bacteriocin-producing host bacteria to the fermentation reactors, mainly because of their acid production and also because of the nutrient competition with fermentation yeasts. GP-15cin may be added by growing up the host GP-15 to produce GP15cin, and then add the cell-free GP15cin to the system to be treated. It is also possible to add the host bacteria to the system to be treated and use its bacteriocin production directly in situ, but its acid production has to be limited or deleted. Acid deficient host strains thus need to be developed. This is usually done by either screening for naturally derived or artificially introduced genetic mutation. This way the host can produce bacteriocin in situ, but not producing harmful other products (such as acids) to inhibit yeast.

It is also possible to use the same bacteriocin-producing bacteria strain as the phage propagation host, to obtain the simultaneous production of bacteriocin and phage in one culture.

### Medical and Veterinary Applications

GP15cin described herein is a potent bacteriocin and showed extremely broad killing spectrum against a list of bacteria, include multiple species of Lactobacillus, Lactococcus, Weissella, Leuconostoc, Pediococcus, Enterococcus, Streptococcus, Staphylococcus, Enterobacter, Bacillus, and Clostridium. The list is likely to expand with new strains being tested. Relying on its broad killing spectrum, GP15cin has utility as antimicrobials for both human medical and animal veterinary applications. In human clinical settings, a list of “superbugs” that are resistant to current treatment antibiotics are emerging. For example, methicillin resistant Staphylococcus aureus (MRSA), vancomycin resistant Enterococcus faecalis, penicillin resistant Pheumococcus, Propionibacterium acne, and Streptococcus mutans are all significant human pathogens, and the list of Gram-positive antibiotic-resistant strains is expanding. Research data to date indicates that two-peptide lantibiotics may be employed, either alone or in combination with other lantibiotics/cell membrane-acting agents, as an effective therapy to control these drug-resistant pathogens (Lawton E, Ross R P, Hill C, Cotter P D; Two-peptide lantibiotics: a medical perspective. Mini-Reviews in Medicinal Chemistry 2007, 7, 1236-1247). Little resistance to current lantibiotics has been observed, highlighting the great potential of their medical applications. GP15cin described herein can be used as a viable alternative to conventional antibiotics to control pathogens in both human medical and animal veterinary applications.

The U.S. Pat. No. 7,666,407 describes the use of a specific bacteriocin for control of dental caries, the disclosure of which is incorporated here by reference for all purposes. The same method for control can be used with GP-15cin.

### Isolation and Characterization of Phage LferInf

### (1) Isolation of Phage LferInf and its Killing Spectrum Against LAB

LAB strains isolated from ethanol fermentation plants were used as host strains for phage isolation and characterization. Using the host strain L. fermentum 0315-25, phage LferInf was isolated from municipal wastewater influent water. The activities of LferInf were tested against LAB strains isolated from different ethanol plants. LferInf showed broad host activities within L. fermentum species. LferInf infected all 12 L. fermentum strains isolated from 8 different plants. LferInf showed activities against some strains of L. mucosae, L. brevis, L. delbueckii, L. reuteri, as well as some undefined Lactobacillus strains.

### (2) Morphological Characterization of Phage LferInf

From transmission electron microscopic (TEM) images, LferInf was observed to belong to Myoviridae family with a contractile tail. The capsid of LferInf was determined to be 88.8 nm (±3.0 nm) in diameter. The tail is 201.7 nm (±4.3 nm) in length and 20.4 nm (±0.9 nm) in width.

### (3) Genomic Characterization of Phage LferInf

Phage LferInf has a genome of 106,071 bp, which carries 124 putative protein-coding genes of more than 40 amino acids each. 95 genes were detected in the plus strand and 39 on the minus strand, with 48 genes encoding hypothetical novel proteins with no matches detectable by BlastP in the NCBI nr database. Two tRNAs are adjacent and located on the minus strand. LferInf genome has a GC content of 38.16%.

Several major functional protein modules were identified in the LferInf genome. These include modules responsible for lysis, DNA packaging, head and tail morphogenesis, and DNA replication. Based on its genome features, it was determined that LferInf is one of the SPO-1 like phages in Myoviridae family.

### Application of Phage LferInf in Biofuel Fermentation

Fermentation models simulating bacterial contamination during ethanol fermentation were set up. In these models, yeast was grown on corn mash and the fermentation was challenged with L. fermentum strain 0315-25. The ability of phage to inhibit the contaminating L. fermentum and restore ethanol production by yeast was evaluated. Compared to the infection free control (yeast alone without bacteria), challenging the system with L. fermentum 0315-25 at 10⁷ cfu/ml decreased ethanol yield from 13.7% to 11.7%, increased residual glucose level from 0.44% to 2.87%, increased lactic acid level from 0.19% to 0.53% (w/v), and acetic acid level from 0.08% to 0.28%, in the infection control at the end (72 h) of experiment. Phage treatment mitigated the L. fermentum infection and restored the levels of ethanol to 13.4% (w/v), glucose to 0.04% (w/v), lactic acid to 0.24% (w/v), and acetic acid to 0.08% (w/v). All these levels are comparable to those of the infection free control.

It was clearly demonstrated that adding phage LferInf to L. fermentum-contaminated yeast fermentation models resulted in effective L. fermentum control. Lactobacillus, especially L. fermentum, predominate the contaminating LAB population in commercial ethanol plants. There is great potential of using phage to control Lactobacillus in ethanol fermentation plants. The phage-based products may also be used prophylacticaly in preventing the contaminating LAB from reaching a high level during ethanol fermentation process.

The mixture of one or more phages to the GP-15cin bacteriocin described herein offers a highly effective antimicrobial cocktail for remediation of unwanted bacteria, particularly in ethanol fermentation processes but also in human and animal bacterial infection control.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A composition of matter for remediation of unwanted bacteria comprising a Class 1b bacteriocin that is a two-peptide lantibiotic consisted of the peptide Plwα, comprised of 29 residues, and the peptide Plwβ, comprised of 32 residues, with the genes encoding the precursor proteins being located next to each other in a transcriptional unit that includes additional genes required for lantibiotic biosynthesis.

2. The composition of claim one wherein the Class 1b bacteriocin is isolated from the indigenous lactic acid bacteria L. plantarum contained in corn mash from an ethanol fermentation process.

3. The composition of claim 1 comprising an aqueous solution having a pH range of 4-10 and maintained below 65° C.

4. The composition of claim 1 wherein the Class 1b bacteriocin is derived from over-producing mutants of L. plantarum by natural mutation or genetic manipulation.

5. The composition of claim 1 also comprising bacteriophage that acts synergistically with the Class 1b two-peptide lantibiotic bacteriocin in broadening the scope and increasing the efficiency of killing unwanted bacteria.

6. The composition of claim 5 wherein the bacteriophage LferInf is a bacteriophage that has a genome of 106,071 bp, which carries 124 putative protein-coding genes of more than 40 amino acids each and on which 95 genes were detected in the plus strand and 39 on the minus strand, with 48 genes encoding hypothetical novel and wherein two tRNAs are adjacent and located on the minus strand.

7. A method for treating unwanted bacteria in humans or animals by application of a composition comprising a Class 1b bacteriocin that is a polycyclic two-peptide lantibiotic consisted of the peptide Plwα, comprised of 29 residues, and the peptide Plwβ, comprised of 32 residues, with the genes encoding the precursor proteins being located next to each other in a transcriptional unit that includes additional genes required for lantibiotic biosynthesis.

8. The method of claim 7 wherein the Class 1b bacteriocin is produced from L. plantarum isolated from lactic acid bacteria contained in corn mash from an ethanol fermentation process.

9. The method of claim 7 wherein the Class 1b two-peptide lantibiotic bacteriocin is mixed with a bacteriophage designated LferInf that has a genome of 106,071 bp, which carries 124 putative protein-coding genes of more than 40 amino acids each and on which 95 genes were detected in the plus strand and 39 on the minus strand, with 48 genes encoding hypothetical novel proteins.

10. The method of claim 7 wherein the unwanted bacteria is that bacteria that causes dental caries.

11. A method for the control of unwanted bacteria in a fermentation process by adding to the feed to the process or to the fermentation vessel a composition comprising a Class 1b bacteriocin that is a two-peptide lantibiotic consisted of the peptide Plwα, comprised of 29 residues, and the peptide Plwβ, comprised of 32 residues, with the genes encoding the precursor proteins being located next to each other in a transcriptional unit that includes additional genes required for lantibiotic biosynthesis.

12. The method of claim 11 wherein the Class 1b bacteriocin is contained in an aqueous solution having a pH range of 4-10 and maintained below 65° C.

13. The method of claim 11 wherein the Class 1b bacteriocin is derived from over-producing system mutants of L. plantarum by natural mutation or genetic manipulation.

14. The method of claim 11 wherein the Class 1b two-peptide lantibiotic bacteriocin is produced in situ in the fermentation process by adding to the process feed or fermentation vessel Lactobacillus plantarum bacteria capable of producing the Class 1b bacteriocin virulent for the unwanted bacteria in a sufficient amount and for sufficient time to destroy a detectable number of unwanted bacteria.

15. The method of claim 11 wherein the Class 1b two-peptide lantibiotic bacteriocin is mixed with a bacteriophage designated LferInf that has a genome of 106,071 bp, which carries 124 putative protein-coding genes of more than 40 amino acids each and on which 95 genes were detected in the plus strand and 39 on the minus strand, with 48 genes encoding hypothetical novel proteins.

16. The method of claim 11 wherein the Class 1b two-peptide lantibiotics bacteriocin is added to the fermentation reactor after at least an hour of fermentation has progressed.
